(19) United States
(12) Patent Application Publication
(10) Pub. No.: US 2014/0127188 A1
(43) Pub. Date: May 8, 2014

(54) METHOD FOR TREATMENT OF ADIPOSE DEPOSITS

(71) Applicants: Katherine Cernok, Costa Mesa, CA (US); Kenneth Martinez, Aliso Viejo, CA (US)

(72) Inventors: Katherine Cernok, Costa Mesa, CA (US); Kenneth Martinez, Aliso Viejo, CA (US)

(21) Appl. No.: 14/233,667

(22) PCT Filed: Jul. 19, 2012
(86) PCT No.: PCT/US12/47405

§ 371 Date: Jan. 17, 2014

Related U.S. Application Data
(60) Provisional application No. 61/509,700, filed on Jul. 20, 2011.

(51) Int. Cl. A61K 38/48

(52) U.S. Cl. CPC A61K 38/4893
USPC 424/94.67

(57) ABSTRACT

Embodiments of the invention include methods and compositions for treatment of adipose deposits.

### CROSS REFERENCE TO RELATED APPLICATION

This patent application claims priority pursuant to 35 U.S.C. §119(e) to U.S. Provisional Patent Application Ser. No. 61/509,700 filed Jul. 20, 2011, which is hereby incorporated by reference in its entirety.

### FIELD OF THE INVENTION

The present invention relates to methods for treating adipose deposits using botulinum toxins.

### BACKGROUND

Obesity has become an epidemic in the United States, with 60% of the adult population reported as overweight or obese (Khaitan, 2005). This health problem is emerging in well-developed as well as developing countries (Prentice, 2006). The burden of obesity can lead to further health issues, but also psychological disturbances of embarrassment, discrimination and depression (Villareal, 2005). Numerous therapies exist targeted to reduce overall adipose tissue levels, such as lipase inhibitors and appetite suppressants. Additionally, there are surgical interventions, including bariatric surgery or LAPBAND®, that can reduce food intake leading to reduced total body fat levels.

In recent years, botulinum toxin A has been proposed to reduce adiposity in subcutaneous fat cells (Lim, 2006). The mechanism by which this occurs is unknown, however parasympathetic signaling and disruption of various lipid membrane structures by the toxin can be speculated. Fat distribution is thought to be regulated by neural signaling under autonomic control. Moreover, parasympathetic signaling is shown to be heavily involved in adiposity. Balbo et al. (2000) reported that fat accumulation was decreased in vagotomized obese rats. Fat pad-specific vagotomy is shown to reduce the insulin-dependent uptake of glucose and free fatty acids in adipose tissue (Kreier, 2002). Visceral adiposity is shown to be linked to both high sympathetic and parasympathetic tone (Lindmark, 2005).

### The Clinical Effects of Botulinum Neurotoxin

### Voluntary Motor Nerves

The first and still primary use of botulinum toxin is to block motor nerve communication with muscle fibers. BT is injected within the target muscle. The botulinum toxin is then internalized into motor neurons where it decreases or stops the release of the neurotransmitter acetylcholine (AChE), thereby causing paresis or paralysis of the muscle. Scott introduced the concept of localized muscular injections of botulinum toxin for the specific condition of strabismus (squint, crossed eyes). Later botulinum toxin was found to be particularly useful for movement disorders such as tics, spasms, contractures, cramps and tremors. More recently, the injection of botulinum toxin into facial muscles has been found to ameliorate skin wrinkling and lines related to aging. Another recent application of botulinum toxin injections is to decrease the pain accompanying muscle tension in conditions such as headache and temporomandibular joint syndrome.

### Autonomic Motor Neurons

The autonomic nervous system is divided into a parasympathetic system and a sympathetic system. The parasympathetic neurons use acetylcholine as their neurotransmitter and they can be blocked with botulinum toxin. The sympathetic nervous system uses noradrenaline as its neurotransmitter with the single exception of sweating) and this neurotransmitter is not blocked by botulinum toxin. Effector neurons of the parasympathetic system innervate and control the contraction of smooth muscles. Injections of botulinum toxin have been used to decrease tone in the smooth muscles of the lower esophageal sphincter, esophagus, stomach wall, and pyloric sphincter, sphincter of Odi, anal sphincter, and urinary bladder.

### Autonomic Secretory Neurons

In addition to their innervation of smooth muscle, neurons of the autonomic system control or modulate a wide variety of other functions such as the secretion of various glands throughout the body. botulinum toxin injections have been used to decrease gastric secretions including acid production, nasal and other respiratory secretions, and tearing.

### Neuropeptides

In addition to the neurotransmitters released at localized synaptic sites, many autonomic and sensory nerves can release neuropeptides along part or all of the length of the axons. These peptides are most noticeable in skin as mediators of inflammation, allergic reactions and pain. For example injury in a small area of skin causes reflex vasodilation in surrounding areas. These reactions are neurally mediated and depend on the release of neuropeptides. Although the neurogenic vasodilation of skin is blocked by botulinum toxin, whether other phenomenon such as pain and swelling are blocked is still uncertain.

The mechanism of action for botulinum toxin type A has a direct effect on acetylcholine release, and could thus effect the vagotone at the site of the injected muscle. Additively, studies on the mechanisms of the 7 serotypes of botulinum toxin A-G report on their involvement in disruption of the cell membrane lipid bilayer and in lipid rafts, among other adipocyte fatty structures. Changes in pH, disruption in lipid rafts and formation of ion channels in lipid bilayers have all been published as mechanisms for botulinum toxins (Montecucco, 1989; Petro, 2006; Kamta, 1994; Oblatt-Montal, 1995). Adipocytes are comprised of various types of structures, such as free fatty acids and phospholipids. The toxin may prove to disrupt the adipose tissue at the site of injection, but these mechanisms are currently unknown.

Recently, Bagheri et al. (2010) investigated the lipolytic effect of botulinum toxin by injecting 9 subcutaneuos injections into the abdomens of rabbits with saline and 2 different doses of type A toxin. The group reported both quantitative and qualitative changes in the treatment groups versus placebo when examining excised abdominal fat tissue. Changes in abdominal subcutaneous adipose tissue was observed in thinning of the fat layer, decrease in fat globular size, fragmenting and shattering of fat globules and disappearance of fat globules within the fat cells (Bagheri, 2010). Fat cell volume was reduced by 65% and 77% compared to placebo rabbit abdomen. Fat cell surface was also significantly reduced.

The anaerobic, gram positive bacterium Clostridium botulinum produces a potent polypeptide neurotoxin, botulinum toxin, which causes a neuroparalytic illness in humans and animals known as botulism. The spores of Clostridium botulinum are found in soil and can grow in improperly sterilized and sealed food containers of home based canneries, which are the cause of many of the cases of botulism. The effects of botulism typically appear 18 to 36 hours after eating the foodstuffs infected with a Clostridium botulinum culture or spores. The botulinum toxin can apparently pass unattenuated through the lining of the gut and attack peripheral motor neurons. Symptoms of botulinum toxin intoxication can progress from difficulty walking, swallowing, and speaking to paralysis of the respiratory muscles and death.

Botulinum toxin type A is the most lethal natural biological agent known to man. About 50 picograms of botulinum toxin type A (available from Allergan, Inc., of Irvine, Calif. under the tradename BOTOX®) is a LD₅₀ in mice. One unit (U) of botulinum toxin is defined as the LD₅₀ upon intraperitoneal injection into female Swiss Webster mice weighing 18-20 grams each. Seven immunologically distinct botulinum neurotoxins have been characterized, these being respectively botulinum neurotoxin serotypes A, B, C₁, D, E, F and G, each of which is distinguished by neutralization with type-specific antibodies. The different serotypes of botulinum toxin vary in the animal species that they affect and in the severity and duration of the paralysis they evoke. The botulinum toxins apparently bind with high affinity to cholinergic motor neurons, are translocated into the neuron and block the release of acetylcholine.

Botulinum toxins have been used in clinical settings for the treatment of neuromuscular disorders characterized by hyperactive skeletal muscles. Botulinum toxin type A has been approved by the U.S. Food and Drug Administration for the treatment of blepharospasm, strabismus, hemifacial spasm, cervical dystonia, and migraine headaches. botulinum toxin type B has also been approved by the FDA for the treatment of cervical dystonia. Clinical effects of peripheral intramuscular botulinum toxin type A are usually seen within one week of injection. The typical duration of symptomatic relief from a single intramuscular injection of botulinum toxin type A averages about three months.

It has been reported that botulinum toxin type A has been used in clinical settings as follows:

about 75-125 U of BOTOX® per intramuscular injection (multiple muscles) to treat cervical dystonia;

5-10 U of BOTOX® per intramuscular injection to treat glabellar lines (brow furrows) (5 U injected intramuscularly into the procerus muscle and 10 U injected intramuscularly into each corrugator supercilii muscle);

about 30-80 U of BOTOX® to treat constipation by intrasphincter injection of the puborectalis muscle;

about 1-5 U per muscle of intramuscularly injected BOTOX to treat blepharospasm by injecting the lateral pre-tarsal orbicularis oculi muscle of the upper lid and the lateral pre-tarsal orbicularis oculi of the lower lid.

to treat strabismus, extraocular muscles have been injected intramuscularly with between about 1-5 U of BOTOX®, the amount injected varying based upon both the size of the muscle to be injected and the extent of muscle paralysis desired (i.e. the amount of diopter correction desired).

to treat upper limb spasticity following stroke by intramuscular injections of BOTOX® into five different upper limb flexor muscles, as follows:

(a) flexor digitorum profundus: 7.5 U to 30 U

(b) flexor digitorum sublimus: 7.5 U to 30 U

(c) flexor carpi ulnaris: 10 U to 40 U

(d) flexor carpi radialis: 15 U to 60 U

(e) biceps brachii: 50 U to 200 U.

Each of the five indicated muscles has been injected at the same treatment session, so that the patient receives from 90 U to 360 U of upper limb flexor muscle BOTOX® by intramuscular injection at each treatment session.

To treat migraine, pericranial (symmetrically into glabellar, frontalis and temporalis muscles) injection of 25 U of BOTOX® has showed significant benefit as a prophylactic treatment compared to vehicle as measured by decreased measures of migraine frequency, maximal severity, associated vomiting and acute medication use over the three month period following the 25 U injection.

Additionally, intramuscular botulinum toxin has been used in the treatment of tremor in patients with Parkinson's disease, although it has been reported that results have not been impressive. Marjama-Jyons, J., et al., Tremor-Predominant Parkinson's Disease, Drugs & Aging 16(4); 273-278:2000.

In addition to having pharmacologic actions at the peripheral location, botulinum toxins may also have inhibitory effects in the central nervous system. Work by Weigand et al., Naunyn-Schmiedeberg's Arch. Pharmacol. 1976; 292, 161-165, and Habermann, Naunyn-Schmiedeberg's Arch. Pharmacol. 1974; 281, 47-56 showed that botulinum toxin is able to ascend to the spinal area by retrograde transport. As such, a botulinum toxin injected at a peripheral location, for example intramuscularly, may be retrograde transported to the spinal cord.

U.S. Pat. No. 5,989,545 discloses that a modified clostridial neurotoxin or fragment thereof, preferably a botulinum toxin, chemically conjugated or recombinantly fused to a particular targeting moiety can be used to treat pain by administration of the agent to the spinal cord.

A botulinum toxin has also been proposed for the treatment of rhinorrhea, hyperhydrosis and other disorders mediated by the autonomic nervous system (U.S. Pat. No. 5,766,605), tension headache, (U.S. Pat. No. 6,458,365), migraine headache (U.S. Pat. No. 5,714,468), post-operative pain and visceral pain (U.S. Pat. No. 6,464,986), pain treatment by intraspinal toxin administration (U.S. Pat. No. 6,113,915), Parkinson's disease and other diseases with a motor disorder component, by intracranial toxin administration (U.S. Pat. No. 6,306,403), hair growth and hair retention (U.S. Pat. No. 6,299,893), psoriasis and dermatitis (U.S. Pat. No. 5,670,484), injured muscles (U.S. Pat. No. 6,423,319, various cancers (U.S. Pat. No. 6,139,845), pancreatic disorders (U.S. Pat. No. 6,143,306), smooth muscle disorders (U.S. Pat. No. 5,437,291, including injection of a botulinum toxin into the upper and lower esophageal, pyloric and anal sphincters)), prostate disorders (U.S. Pat. No. 6,365,164), inflammation, arthritis and gout (U.S. Pat. No. 6,063,768), juvenile cerebral palsy (U.S. Pat. No. 6,395,277), inner ear disorders (U.S. Pat. No. 6,265,379), thyroid disorders (U.S. Pat. No. 6,358,513), parathyroid disorders (U.S. Pat. No. 6,328,977). Additionally, controlled release toxin implants are known (see e.g. U.S. Pat. Nos. 6,306,423 and 6,312,708).

### SUMMARY

Embodiments of the invention include a method of treating adipose deposits comprising administering a botulinum composition to the affected area of the patient. In embodiments the patient is female. In embodiments the botulinum is botulinum type A administered in an amount between 50 and 300 units.

### DESCRIPTION OF EXEMPLARY EMBODIMENTS

Embodiments of the invention utilize methods and compositions comprising botulinum toxins and like substances to treat, for example, adipose deposits such as, for example, cellulite, lipoma, and the like. In certain embodiments, the method of administration can be systemic, such as, for example, intravenously, or local, such as, for example, via injection, implant, a topical formulation, or the like.

In one aspect, the invention comprises a method of treating adipose deposits, comprising administering an effective amount of a botulinum toxin composition to or below the skin of a patient in need thereof. In certain embodiments, for example, the botulinum toxin composition comprises botulinum toxin encapsulated in phospholipid micelles, and/or one or more primary stabilizers, and/or one or more skin penetration enhancers. In an alternate embodiment, injection of botulinum toxin A is carried out at multiple sites in the skin, wherein the sites of adjacent injections are separated by about 0.1 to 10 cm., or by about 0.5 to about 5 cm. or by about 1.5 to about 3 cm. The toxins may be any of the botulinum toxins A,B,C,D,E,F or G. The amounts administered may vary between 0.1 and 1000 U, suitably about 1 to about 40, often from about 5 to about 10 U, depending on the manufactures specifications, the class of the toxin and the mode of administration. Thus 1 U of Botox equals about 2-4 units of Dysport and about 20-40 units of Myobloc. The distances between injections can vary from about 1 mm to about 10 cm, or from about 5 mm to about 5 cm, or from about 1 cm to about 3 cm. Thus for example botulinum A may be suitably administered by intradermal injection between about 0.1 to about 10 U at a separation of from about 0.5 to about 10 cm. preferably at about 2.5 cm. Botulinum B can be administered in the range of 1-500 U, preferably 100 U separated by 1.5 cm.

The following definitions apply herein:

“About” means approximately or nearly and in the context of a numerical value or range set forth herein means .+−.10% of the numerical value or range recited or claimed.

“Affliction” includes a disease, disorder, problem and/or a cosmetically undesirable state or condition in an individual.

“Alleviating” means a reduction in the occurrence of a symptom related to adipose deposits. Thus, alleviating includes some reduction, significant reduction, near total reduction, and total reduction of a symptom related to adipose deposits. An alleviating effect may not appear clinically for between 1 and 7 days after administration of a botulinum toxin to a patient.

“Botulinum toxin” means a botulinum neurotoxin as either pure toxin (i.e. about 150 kDa weight molecule) or as a complex (i.e. about 300 to about 900 kDa weight complex comprising a neurotoxin molecule and one or more associated non-toxic molecules), and excludes botulinum toxins which are not neurotoxins such as the cytotoxic botulinum toxins C2 and C3, but includes recombinantly made, hybrid, modified, and chimeric botulinum toxins.

“Effective amount” as applied to the neurotoxin means that amount of the neurotoxin generally sufficient to effect a desired change in the subject. In some embodiments, the neurotoxin can be administered in an amount between about 0.01 U/kg and about 35 U/kg and the pain treated can be substantially alleviated for between about 1 month and about 27 months, for example for from about 1 month to about 6 months.

“Improved patient function” means an improvement measured by factors such as reduced pain, reduced time spent in bed, smoother skin, fewer skin irregularities, increased ambulation, healthier attitude, more varied lifestyle and/or healing permitted by normal muscle tone. Improved patient function is synonymous with an improved quality of life (QOL). QOL can be assessed using, for example, the known SF-12 or SF-36 health survey scoring procedures. SF-36 assesses a patient's physical and mental health in the eight domains of physical functioning, role limitations due to physical problems, social functioning, bodily pain, general mental health, role limitations due to emotional problems, vitality, and general health perceptions. Scores obtained can be compared to published values available for various general and patient populations.

“Local administration” or “locally administering” means administration (i.e. by a subcutaneous, intramuscular, subdermal or transdermal route) of a pharmaceutical agent to or to the vicinity of a dermal or subdermal location of a patient. Appropriate methods of local administration can include, for example, injection, topical application, implant, or the like.

“Treating” means to alleviate (or to eliminate) at least one symptom related to adipose deposits, such as skin irregularity, for example irregularities in skin contour, texture, or topography, either temporarily or permanently.

The pharmaceutical compositions contemplated by this invention include pharmaceutical compositions suited for topical and local action.

The term “topical” as employed herein relates to the use of a composition, as described herein, incorporated in a suitable pharmaceutical carrier, and applied at the site of an adipose deposit or deposits for exertion of local action. Accordingly, such topical compositions include those pharmaceutical forms in which the compound is applied externally by direct contact with the skin surface to be treated. Conventional pharmaceutical forms for this purpose include ointments, liniments, creams, shampoos, lotions, pastes, jellies, sprays, aerosols, and the like, and can be applied in patches or impregnated dressings depending on the part of the body to be treated. The term “ointment” embraces formulations (including creams) having oleaginous, water-soluble and emulsion-type bases, e.g., petrolatum, lanolin, polyethylene glycols, as well as mixtures of these.

The compositions can be applied applied a single time or repeatedly at regular or non-regular intervals for a sustained period of time. In certain embodiments, compositions of the invention can be administered topically on the part of the body to be treated, for example, the thighs, buttocks, legs, etc. In certain embodiments, compositions of the invention can be administered systemically, such as, for example, intravenously.

For topical use on the body, the compositions can be formulated in aqueous solutions, creams, ointments or oils exhibiting physiologically acceptable osmolarity by addition of pharmacologically acceptable buffers and salts. Such formulations may or may not, depending on the dispenser, contain preservatives such as benzalkonium chloride, chlorhexidine, chlorobutanol, parahydroxybenzoic acids and phenylmercuric salts such as nitrate, chloride, acetate, and borate, or antioxidants, as well as additives like EDTA, sorbitol, boric acid etc. as additives. Furthermore, particularly aqueous solutions may contain viscosity increasing agents such as polysaccharides, e.g., methylcellulose, mucopolysaccharides, e.g., hyaluronic acid and chondroitin sulfate, or polyalcohol, e.g., polyvinylalcohol. Various slow releasing gels and matrices may also be employed as well as soluble and insoluble ocular inserts, for instance, based on substances forming in-situ gels. Depending on the actual formulation and compound to be used, various amounts of the drug and different dose regimens may be employed.

The botulinum toxin can be selected from the group consisting of botulinum toxin types A, B, C, D, E, F and G. Botulinum toxin type A is a preferred botulinum toxin.

Botulinum toxins for use according to the present invention can be stored in lyophilized, vacuum dried form in containers under vacuum pressure or as stable liquids. Prior to lyophilization the botulinum toxin can be combined with pharmaceutically acceptable excipients, stabilizers and/or carriers, such as albumin. The lyophilized material can be reconstituted with saline or water to create a solution or composition containing the botulinum toxin to be administered to the patient.

Exemplary, commercially available, botulinum toxin containing compositions include, but are not limited to, BOTOX® (Botulinum toxin type A neurotoxin complex with human serum albumin and sodium chloride) available from Allergan, Inc., of Irvine, Calif. in 100 unit vials as a lyophilized powder to be reconstituted with 0.9% sodium chloride before use), DYSPORT® (Clostridium Botulinum type A toxin haemagglutinin complex with human serum albumin and lactose in the formulation, available from Ipsen Limited, Berkshire, U.K. as a powder to be reconstituted with 0.9% sodium chloride before use) which can be used at about 3 to about 4 times the amounts of BOTOX® as set forth herein in each instance, and MYOBLOC® (an injectable solution comprising botulinum toxin type B, human serum albumin, sodium succinate, and sodium chloride at about pH 5.6, available from Solstice Neurosciences, Inc., South San Francisco, Calif.) which can be used at about 30 to about 50 times the amounts of BOTOX® as set forth herein in each instance, as known in the art. XEOMIN® (a 150 kDa botulinum toxin type A formulation available from Merz Pharmaceuticals, Potsdam, Germany) is another useful neurotoxin which can be used at about 1 to about 2 times the amounts of BOTOX® as set forth herein in each instance.

In additional embodiments, no less than about 10 U and no more about 400 U of BOTOX®; no less than about 30 U and no more than about 1600 U of DYSPORT®, and; no less than about 250 U and no more than about 20000 U of MYOBLOC® are administered per site, per patent treatment session.

In still further embodiments, no less than about 20 U and no more about 300 U of BOTOX®; no less than about 60 U and no more than about 1200 U of DYSPORT®, and; no less than about 1000 U and no more than about 15000 U of MYOBLOC® are administered per site, per patent treatment session.

Although the composition may only contain a single type of botulinum toxin, such as, for example, type A, as the active ingredient, other therapeutic compositions may include two or more types of botulinum toxins. For example, a composition administered to a patient may include botulinum toxin type A and botulinum toxin type B. Administering a single composition containing two different neurotoxins can permit the effective concentration of each of the neurotoxins to be lower than if a single neurotoxin is administered to the patient while still achieving the desired therapeutic effects. The composition administered to the patient may also contain other pharmaceutically active ingredients, such as, for example, protein receptor or ion channel modulators, or the like, in combination with the neurotoxin or neurotoxins.

In certain embodiments, compositions of the invention can comprise re-targeted endopeptidases, molecules derived by replacing the naturally-occurring binding domain of a clostridial toxin with a targeting domain showing a selective binding activity for a non-clostridial toxin receptor present in a cell of interest. Such modifications to the binding domain result in a molecule that is able to selectively bind to a non-clostridial toxin receptor present on the target cell. A re-targeted endopeptidase can bind to a target receptor, translocate into the cytoplasm, and exert its proteolytic effect on the SNARE complex of the neuronal or non-neuronal target cell of interest.

Certain embodiments of the invention can utilize an implant for administration. Implants useful in practicing the methods disclosed herein may be prepared by mixing a desired amount of a stabilized Botulinum toxin (such as non-reconstituted BOTOX®) or re-targeted endopeptidase into a solution of a suitable polymer dissolved in methylene chloride. The solution can be prepared at room temperature. The solution can then be transferred to a Petri dish and the methylene chloride evaporated in a vacuum desiccator. Depending upon the implant size desired and hence the amount of incorporated drug, a suitable amount of the dried neurotoxin-incorporating implant is compressed at about 8000 p.s.i. for 5 seconds or at 3000 p.s.i. for 17 seconds in a mold to form implant discs encapsulating the neurotoxin. See e.g. Fung L. K. et al., Pharmacokinetics of Interstitial Delivery of Carmustine 4-Hydroperoxycyclophosphamide and Paclitaxel From a Biodegradable Polymer Implant in the Monkey Brain, Cancer Research 58;672-684:1998.

Additionally, in some embodiments, a physician can alter the dosage in each case in accordance with the assessment of the severity of the condition, as typically done when treating patients with a condition or disorder. Further, in some embodiments, the treatment may have to be repeated at least one additional time, in some cases several times, depending on the severity of the condition and the patient's overall health. If, for example, a patient is not deemed physically suitable for a full administration of botulinum toxin, or if a full administration is not desired for any reason, smaller doses on multiple occasions may prove to be efficacious.

Of course, an ordinarily skilled medical provider can determine the appropriate dose and frequency of administration(s) to achieve an optimum clinical result. That is, one of ordinary skill in medicine would be able to administer the appropriate amount of the toxin, for example botulinum toxin type A, at the appropriate time(s) to effectively treat the disorder. The dose of the neurotoxin to be administered depends upon a variety of factors, including the severity of the eye disorder. The dose of the toxins employed in accordance with this invention may be equivalent to the dose of BOTOX® used in accordance with the present invention described herein. In various methods of the present invention, from about 0.01 U/kg (U of botulinum toxin per kilogram of patient weight) to about 15 U/kg, of BOTOX® can be administered. In some embodiments, about 0.1 U/kg to about 20 U/kg of BOTOX® can be administered. Use of from about 0.1 U/kg to about 30 U/kg of a BOTOX®, is within the scope of a method practiced according to the present disclosed invention. In one embodiment, about 0.1 U/kg to about 150 U/kg botulinum toxin, for example type A, may be administered.

Significantly, a method within the scope of the present invention can provide improved patient function.

The botulinum toxin composition can be administered at, for example, a dose of 10 to 1000 U of botulinum toxin per affected site, or a dose of 20 to 800 U of botulinum toxin per affected site, or a dose of 50 to 500 U of botulinum toxin per affected site, or a dose of 100 to 400 U of botulinum toxin per affected site, or a dose of 200 to 300 U of botulinum toxin per affected site, or the like.

In some embodiments, the affected area can comprise multiple toxin administration sites.

In some embodiments the botulinum toxin composition can be administered at, for example, a dose of 0.01 to 20 U of botulinum toxin per administration site, or a dose of 0.05 to 15 U of botulinum toxin per administration site, or a dose of 0.1 to 10 U of botulinum toxin per administration site, or a dose of 1 to 5 U of botulinum toxin per administration site, or amounts within those ranges, or the like. In certain embodiments, administration can continue, for example, for a period of 1 day to 8 weeks. In an embodiment, the botulinum toxin composition is administered at a dose of 0.1 to 10 U of botulinum toxin per administration site per day for a duration of 1 to 2 weeks. In another embodiment, the botulinum toxin composition is administered at a dose of 1 to 5 U for duration of 2 days to 10 days. In yet another embodiment, the botulinum toxin composition is administered at a dose of 2 to 3 U of botulinum toxin per affected site per day for a duration of 5 to 7 days.

Suitable active ingredients for inclusion in the composition include botulinum toxin type A, type B, type C, type D, type E, type F, and type G. Other active ingredients can include, but are not limited to androgens, androstenediol and androisoxazole (for anabolic disorders), testosterone (hypogonadism, muscle wasting, male impotence, postmenopausal symptoms in women), dehydrotestosterdne (hypogdnadism, muscle wasting), dehydroepiandrostenone (muscle wasting, fat reduction, fitness); estrogens (postmenopausal symptoms, birth control), 17 betaestradiol, estradiol-3,17-diacetate, estradiol-3-acetate, estradiol-17-acetate, estradiol-3, 17-valerate, estradiol-3-valerate, estradiol-17-valerate, ethinyl estradiol, estrone; progesterones (prevent endometriosis, prevent endometrial cancer, control habitual abortion, suppress or synchronize estrus, promote hair growth), progesterone (preg-4-ene-3,20-dione), norethindrone, norgestrieone, norgestadienone, norgestrel, norgestimate, progestogenic acid, dihydroprogesterol, nomagesterol. The testosterone hormone may be used in any of its usual forms, such as, acetate, propionate, 17-beta-cyclopentanepropionate, enanthanate, isobutyrate, undeconate, and the like. Similarly, the estradiols may additionally be used in any of the known or newly developed forms, such as, for example, pivalate, propionate, cypionate, benzoate and other esters.

Compositions of the invention can also include insulin, insulin like growth factors, vaccines, glucagon-like peptide (GLP), Insulin-like Growth Factor (IGF), heparin, hirugen, hirulos, huridine, mumps, measles and rubella vaccine, typhoid vaccine, hepatitis A vaccine, hepatitis B vaccine, herpes simplex virus, bacterial toxoids, cholera toxin B-subunit, influenza vaccine virus, bordetala pertussis, vaccinia virus, adenovirus, canary pox, polio vaccine virus, plasmodium falciparum, bacillus calmette geurin (BCG), klebsiella pneumoniae, HIV envelope glycoproteins, bovine somatropine, estrogens, androgens, insulin growth factors, interleukin-1, interleukin-I1 and cytokins, small molecule drugs such as NSAIDs, narcotics, and various other peptides, genetic material and small molecules.

Other active ingredients can include various types of anti-oxidants. Some examples of free radical scavengers that may be included in the composition include, but are not limited to, Vitamins A, C and E, the minerals Zinc and Selenium, lycopene, the amino acid N-Acetyl-Cysteine and natural plant extracts of Grape Skin, Bilberry and Green Tea.

In certain embodiments, compositions of the invention can include agents that promote healing. For example, vasodilators, such as nitroglycerin and glycerin mononitrate can be encapsulated in a phospholipid micelle and then combined with collagen and/or elastin in a lotion or cream formulation and applied to the skin. Without being limited by the explanation, it is thought that the formulation of vasodilators in the composition enhances the rate of penetration as compared to administration via, for example, a skin patch. Inclusion of hydrogen peroxide and/or a perfluorocarbon may further enhance oxygenation and healing.

The composition can contain a single active ingredient or multiple active ingredients in the same composition. Various combinations of active ingredients are contemplated for inclusion in the composition.

Skin penetration enhancers that promote the absorption of an active ingredient by the skin can also be included in the composition. In particular, one or more skin penetration enhancers may be used to facilitate the permeation of botulinum toxin through the patient's skin. Examples of skin penetration enhancers include, but are not limited to, alcohols, such as short chain alcohols, long chain alcohols, or polyalcohols, amines and amides, such as urea, amino acids or their esters, amides, AZONE®, derivatives of AZONE®, pyrrolidones, or derivatives of pyrrolidones; terpenes and derivatives of terpenes; fatty acids and their esters; macrocyclic compounds; tensides; or sulfoxides such as, decylmethylsulfoxide. Liposomes, transfersomes, lecithin vesicles, ethosomes, water surfactants, such as anionic, cationic, and nonionic surfactants, polyols, and essential oils can also function as skin penetration enhancers.

Embodiments of the invention can comprise phospholipid micelles. In certain embodiments, the phospholipid micelles may comprise sphingosine and cerebroside, for example, or the like. In some embodiments the primary stabilizers may comprise elastin and collagen, for example, or the like. In some embodiments, the one or more skin penetration enhancers can be selected from the group that includes, for example, d-limonene, allantoin, fulvic acid, myrrh, hydroquinone glyquin, quillaja saponaria (QTS), acanthophyllum squarrusom (ATS), either singularly or in combination, or the like.

In an embodiment, the botulinum toxin composition comprises:

approximately 1 to 40% w/w collagen;

approximately 1 to 40% w/w elastin;

approximately 0.1 to 15% w/w sphingosine phospholipid; and

approximately 0.1 to 15% w/w cerebroside phospholipid.

The composition may also be used for topical administration in a format whereby the composition penetrates the skin and transdermally denervates an underlying muscle.

The composition may include d-limonene to enhance penetration of the active ingredient through the dermal layer. Limonene has been found to be an effective skin penetration enhancer at 0.30%, enhancing skin permeation of botulinum toxin Type A approximately fourfold.

Quillaja saponaria (QTS) and Acanthophyllum squarrusom (ATS) total saponins are two natural skin penetration enhancers that may also be included in the composition. They demonstrate moderate activity as skin penetration enhancers.

Allantoin may also be included in the composition. Allantoin acts as a skin protectant and a mild neutral skin penetration enhancer.

Fulvic acid may also be included in the composition as a skin penetration enhancer. Fulvic acid is a low molecular weight antioxidant that enhances the body's absorption of drugs through the transdermal route.

Myrrh may also be included in the composition as a skin penetration enhancer. Myrrh is a gum resin extracted from Arabian and Somolian shrubs.

Eldopaque or hydroquinone glyquin may also be included as skin penetration enhancers.

The use of collagen in the composition, in combination with elastin and a mixture of sphingosine and cerebroside, maintains the integrity of the complex without denaturing or fragmentation or detoxification. Thus, botulinum toxin can be stabilized and the stabilized toxin can be successfully delivered transdermally to achieve similar results to those obtained by intramuscular injection of botulinum toxin. The formulation can be applied all over the face, neck, axillae and hands as opposed to a botulinum toxin injection which is targeted primarily to areas around eyes and the forehead to reduce the wrinkle.

The composition can include microspheres. The composition be a cosmetic composition that includes water and other additives that are normally used in cosmetics. For example, it may include thickening agents, preservatives, emulsifiers, perfumes, dyes or coloring, vegetable or mineral oil, antiseptic agents, acidifying or alkalizing agents, vitamins, anti-UV agents, surfactant, solvents, pH stabilizing agents, and other active ingredients known to be effective on the skin. The composition may also be included in a make-up formulation, such as, for example, a skin foundation or the like.

Additional components can be included to formulate the composition into other formats, such as a cream, lotion, spray, mask, gel, etc., that is suitable for topical administration. If formulated as a cream or a solution, the composition should contain the active ingredient in sufficiently concentrated quantities in order that the composition does not drip off the area of administration.

The composition may also be provided on a patch that is adhesively secured to the skin so that the active ingredient, such as botulinum toxin, can pass from the patch through the skin.

A preferred method for preparing a stabilized botulinum toxin composition for topical application is shown below. Briefly, equal amounts of collagen and elastin are solubilized in saline. In a separate flask, equal amounts of sphingosine and cerebroside are dissolved in alcohol. The alcohol is then removed. Botulinum toxin A is dissolved in saline and then added to the flask and the flask is swirled to coat the botulinum toxin protein with a phospholipid micelle coating. This solution is then added to the solution of collagen and elastin. This method can be used to prepare compositions containing other types of botulinum toxin.

The skin penetration enhancers may be included at various stages of the fabrication process. For example, they may be added to the stabilized composition at the same time as the micellar composition is added to the collagen and elastin mixture. Preferably, the skin penetration enhancers are introduced during the formulation of the stabilized composition into a pharmaceutical or cosmetic formulation.

Any feature or combination of features described herein are included within the scope of the present invention provided that the features included in any such combination are not mutually inconsistent as will be apparent from the context, this specification, and the knowledge of one of ordinary skill in the art.

Of course, an ordinarily skilled medical provider can determine the appropriate dose and frequency of administration(s) to achieve an optimum clinical result. That is, one of ordinary skill in medicine would be able to administer the appropriate amount of the botulinum toxin, for example botulinum toxin type A, at the appropriate time(s) to effectively treat the adipose deposit. The dose of the neurotoxin to be administered depends upon a variety of factors, including the severity of the adipose deposits. The present invention also provides compositions for treating an adipose deposit in a mammal.

It is known that botulinum toxin type A can have an efficacy for up to 12 months (European J. Neurology 6 (Supp 4): S111-S1150:1999), and in some circumstances for as long as 27 months. The Laryngoscope 109:1344-1346:1999. However, the usual duration of an intramuscular injection of botulinum type A is typically about 3 to 4 months.

### EXAMPLES

### Example 1

### Preparation of Botulinum Toxin Compositions

Botulinum toxin (BOTOX®) vials are reconstituted in sterile saline solution (0.9%). The vials are gently shaken to dissolve the botulinum toxin. The reconstituted vials are kept refrigerated and are utilized within 1 hour of reconstitution.

In a round bottom flask of 50 mL capacity, 10 mg of soluble collagen and 10 mg of elastin are combined. The mixture is solubilized in 10 mL of sterile saline solution (0.9%) with continuous stirring. In a separate 50 mL round bottom flask, 5 mg of sphingosine and 5 mg cerebroside are combined. This mixture is dissolved in 1 mL pure ethanol. The alcohol is completely removed by rotary vacuum evaporation to obtain a uniform coating of the sphingosine and cerebroside on the flask wall. To this flask 800 units of botulinum toxin solution in 6 ml of (0.9%) saline is added. The flask is swirled and then stirred continuously for 5 minutes at room temperature to uniformly coat the botulinum toxin with the sphingosine and cerebroside micelle coating. This coated and preserved micellar botulinum toxin solution is then added to the flask containing the mixture of collagen and cross-linked, low molecular weight elastin. The solution is stirred for about 5 minutes and then kept at room temperature in a brown glass vial.

### Example 2

### Botulinum Toxin Cream Formulation

The stabilized botulinum toxin composition of Example 1 is formulated into a cream for topical administration as outlined below. Total volume of the cream (400 mL):

Phase A: De-ionized Water 74.7% Tetra Sodium EDTA 0.5-0.7% Methyl Paraben 0.2% Propylene Glycol 3.0%-4.0% Glycerin 3.0%-4.0%

Phase B: Cetyl Alcohol (Ado 1 52 NE) 2.0% Cetearyl Alcohol 2.0% Glyceryl Stearate 2.0% PEG-100 Stearate 1-2% Stearic Acid (Emersol 132) 4.5% Sorbitan Palmitate 0.5-0.7% Polysorbate-85 1.0% Polysorbate 60 0.5-1% Lanolin Alcohol (Ritachol) 1.0% HoHoba Oil 0.5-1% Lanolin 1-2% Tocopheryl Acetate 0.5-1% Dimethicone 200 0.7-1.0% BHA 0.1% Propylparaben 0.1% Diazolidinyl UREA 0.2%

Phase C: Fragrance (lilac, jasmine) as needed Aloe Vera (powder) 1.5%-2.0% CoQ-10 0.5% Retinyl A 0.03-0.05% Hyaluronic Acid (pure) 1.0-1.5% Talcum Powder (Ti02) 1.0-1.5%

Phase D: d-limonene 0.7% Allantoin 0.5% Fulvic Acid 0.5% Quillaja saponaria (QTS) 0.3% Acanthophyllum squaimsom (ATS) 0.3% Myrrh Extract 0.2% Hydroquinone Glyquin 4.0%

Phase E: Stabilized Botulinum Toxin in Collagen Matrix 800 units.

Procedure: Heat Phase A and Phase B separately with agitation to 75° C. Add Phase A to Phase B and mix 30 minutes at 75° C. Cool down to 20-22° C. and then add Phase C, D and E and continue to agitate until homogenous and one phase.

### Example 3

### Preparation of Botulinum Toxin Micellar Solution

Botulinum toxin A (Botox®, Allergan) is obtained as a lyophilizate and reconstituted in sterile saline solution (0.9%). The vials are shaken to dissolve the botulinum toxin (BOTOX®). The reconstituted vials are kept refrigerated and were utilized, as described below, within 1 hour of reconstitution.

In a round bottom flask of 50 mL capacity, 10 mg of soluble collagen, 10 mg of elastin are weighed. The mixture is solubilized in 10 mL of sterile saline solution (0.9%). The mixture is stirred continuously.

In a separate 50 mL round bottom flask, 5 mg of phosphatidylcholine and 5 mg of cholesterol are weighed. The mixture is dissolved in approximately 1 mL of 70% ethanol. To this solution, 10 mg sodium dodecyl sulfate (SDS) and 10 mg dimethyl sulfoxide (DMSO) are added and solubilized to make a uniform homogenous solution. To this flask 1000 units of botulinum toxin (Botox®) solution in 5 ml of (0.9%) saline is added. The flask is swirled and then stirred continuously for 5 minutes at room temperature. The solution of collagen and elastin is added to this solution. The solution is stirred for about 5 minutes slowly and then kept at room temperature in a brown glass vial.

Preparation of Topical Cream

A topical base composition is prepared as noted below.

Composition of topical base ingredients:

Glycerin (humectant) 4.00 g

Aluminum Zirconium Complex or Alumimum 10.0 g

Chlorohydrate (gelling agent)

Hexamethylene tetraamine (skin penetration enhancer 8.00 and coating agent, and gel forming compound)

Sodium lauryl sulfate 0.10 g

DMSO 0.10 Alcohol Denat. 5.00 g

DMDM Hydantoin (preservative) 0.20 g

Baby powder (fragrance) 0.10 g

Carbopol 940 polymer or hydroxy propyl cellulose 5.00 g

To this base composition is added the stabilized botulinum toxin composition prepared as described above in part (a) in order to achieve a concentration of botulinum toxin type A of 2 Units per 1 mL of resultant cream. The resultant cream is stirred slowly for about 30-45 minutes, to obtain a homogeneous composition. The cream prepared in this manner is then stored at or below room temperature for future use.

### Example 4

### Injection Administration to Treat Cellulite

200-300 Units injected into the subcutaneous fat of obese individual.

A 42 year old female patient with over 2 decades of reported obesity complains of severe lethargy and depression. She is no longer able to perform activities that she used to and has tried numerous pills and therapies to try and reduce her weight. She complains to the physician about the cellulite under her gluteus maximus, of which the physician determines to be the gluteal folds. The circumference of her waist is measured at 60 inches. She is treated with botulinum neurotoxin type A using a 4 mL dilution per 100 units (increases diffusion). She is injected as follows:

the gluteal folds is are divided into a grid of 4×6 squares starting just below the buttocks.

120 Units are administered into 24 injection sites, approximately 5 Units of botulinum neurotoxin type A per injection site on both legs, totalling 240 Units for both legs. botulinum neurotoxin type A is targeted to the subcutaneous adipose tissue. She returns 4 weeks later and reports decreased appearance of cellulite. She is intrigued by the results and has requested to be retreated at the 3 month time period.

Overall, by targeting botulinum neurotoxin type A to the subcutaneous adipose tissue of obese adults, this novel injection paradigm results in a lipolytic effect of injected and adjacent adipose tissue. With retreatment, this adiposity will continue to thin and shatter fat globules in subcutaneous fat and fat cells/adipocytes.

### Example 5

### Topical Administration to Treat Lipoma

50-100 U administered topically into the fatty tissue of a lipoma.

A 58 year old female patient comes to see the physician because there is a rather large lipoma that has reappeared in the space between her clavicle and neck. It is uncomfortable to the patient and since surgical removal was not permanent, she is interested in other options. The physician treats the lipoma with botulinum neurotoxin type A cream. Because this is the first treatment, 50 U is utilized in one administration directly into the lipoma. The patient is examined 4 weeks later and a significant reduction in the size of the lipoma is noted. The physician decides to retreat this patient with another 50 U after 3 months.

What is claimed is:

1. A method of treating an adipose deposit, comprising administering a botulinum composition to the affected area of the patient, thereby treating the adipose deposit.

2. The method of claim 1, wherein the patient is female.

3. The method of claim 1, wherein the botulinum composition comprises botulinum type A.

4. The method of claim 3, wherein the botulinum type A is administered in an amount between about 200 and about 300 units.

5. The method of claim 4, wherein the botulinum toxin is re-administered between about every 2 to about every 6 months.

6. The method of claim 1 wherein the administration comprises topical administration.

7. The method of claim 6 wherein the botulinum composition comprises botulinum type A.

8. The method of claim 1 wherein the administration comprises administration via injection.

9. The method of claim 8 wherein the botulinum composition comprises botulinum type A.

10. The method of claim 9 wherein the botulinum type A comprises the 900 kD type A complex.
